(19) United States
(12) Patent Application Publication
(10) Pub. No.: US 2024/0294932 A1
(43) Pub. Date: Sep. 5, 2024

(54) ELIMINATION OF AFLATOXIN VIA AFLATOXIN-DEGRADING ENZYME EXPRESSION IN CROPS

(71) Applicant: ARIZONA BOARD OF REGENTS ON BEHALF OF THE UNIVERSITY OF ARIZONA, Tucson, AZ (US)

(72) Inventor: Monica Schmidt, Tucson, AZ (US)

(21) Appl. No.: 18/553,953

(22) PCT Filed: Apr. 5, 2022
(86) PCT No.: PCT/US22/23441

§ 371 Date: Oct. 4, 2023

Related U.S. Application Data
(60) Provisional application No. 63/170,777, filed on Apr. 5, 2021.

(51) Int. Cl. C12N 15/82; C12N 9/58

(52) U.S. Cl. CPC C12N 15/8243; C12N 9/58; C12N 15/8234

(57) ABSTRACT

Maize is vital to both US agriculture and the economy. The US provides over half of the maize global market. In the US, field maize production is a $75B endeavor and comprises 95% of the total US grain production. Worldwide there is a net loss of 16 million tons of maize due to aflatoxin contamination. In the US alone, aflatoxin contamination of food/feed results in an estimated $52M-$1.68B agricultural loss every year. Aflatoxin contamination in crops, and subsequently livestock, threatens greater agricultural development, food security and human health. Elimination of aflatoxin is a critical economic and health issue in the US. The present invention features composition and methods for the degradation of aflatoxin in crops post-harvest.

### CROSS-REFERENCES TO RELATED APPLICATIONS

This application claims benefit of U.S. Provisional Application No. 63/170,777 filed Apr. 5, 2021, the specification of which is incorporated herein in its entirety by reference.

### STATEMENT REGARDING FEDERALLY SPONSORED RESEARCH OR DEVELOPMENT

This invention was made with government support under Grant No. 2019-67017-29644, awarded by USDA/NIFA. The government has certain rights in the invention.

### REFERENCE TO A SEQUENCE LISTING

Applicant asserts that the information recorded in the form of an Annex C/ST.25 text file submitted under Rule 13ter.1(a), entitled>>>UNIA_21_05_PCT_Sequence_Listing_ST25<<<, is identical to that forming part of the international application as filed. The content of the sequence listing is incorporated herein by reference in its entirety.

### FIELD OF THE INVENTION

The present invention relates to aflatoxins, which are secondary metabolites produced by certain species of Aspergillus (e.g., A. flavus, A. parasiticus), more particularly to transgenic plant species, such as maize and peanuts, that express an aflatoxin-degrading enzyme gene.

### BACKGROUND OF THE INVENTION

One quarter (25%) of the world's crops are contaminated with mycotoxin. Mycotoxins are toxic secondary compounds produced by a fungal source and can be responsible for massive agricultural losses world-wide. Aflatoxins, a class of mycotoxins, is produced by certain strains of Aspergillus: A. parasiticus, A. flavus and A. nomius, A. pseudotamarii and A. bombycis, however only two of the strains are associated with the agricultural production of aflatoxins, A. parasiticus and A. flavus. A. flavus has the ability to infect a wide range of plant hosts while A. parasiticus is generally limited to ground crop hosts. In the US, the major commodities that are susceptible to aflatoxins include maize, peanuts, cotton and tree nuts. Aflatoxins are toxic and carcinogenic to both animals and humans. If aflatoxin-contaminated food/feed is ingested, it can result in hepatotoxicity, liver cancer, kwashiorkor and Reye's syndrome.

There are 4 aflatoxins: AFB1, AFB2, AFG1 and AFG2. Structural differences give the compounds their namesake characteristic; that is, under ultraviolet light, AFB1 and AFB2 are blue fluorescent where AFG1 and AFG2 are green fluorescent. AFB1 is the most prevalent aflatoxin and unfortunately is the most toxic. The epoxidation of the unsaturated bond at 8,9 position on the terminal furan ring on AFB1 and AFG1 has been shown to be critical for their carcinogenic potency. The absence of this position on AFB2 and AFG2 renders them nontoxic until they are metabolized into AFB1 or AFG1. When animals consume aflatoxin-contaminated feed they produce milk that is in turn contaminated by aflatoxin M1. Aflatoxin M1 is a metabolite of aflatoxin B1, and is known to be carcinogenic, producing tumors and liver cancer in test animals. Due to the high toxicity of aflatoxins, over 100 countries restrict the level in either food or feed, including the United States. Maize destined for humans and dairy cattle has the tightest limit, at 20 parts per billion (ppb). Maize destined to finish beef cattle can contain the fungal toxin at concentrations up to 300 ppb. The legal limit in milk is 0.05 ppb for aflatoxin M1. To put these numbers into perspective, 1 ppb is equivalent to a single drop of water in a 21,700 gallon (82,135 liter) swimming pool or from a time perspective, 1 sec in 31.7 yrs.

Current aflatoxin prevention mechanisms are inadequate. Breeding for fungal resistant crops, agronomic practices to lower the ability for the fungus to grow, biocontrol with atoxigenic Aspergillus strains, improved storage methods post-harvest and using trapping agents to block uptake of aflatoxins are all currently used and still the US sufferers millions in crop losses each year to aflatoxin contamination. Aflatoxin contamination occurs during both pre-harvest conditions while crops are actively growing and in post-harvest storage, so there needs to be effective strategies under both conditions. Biotechnology is a viable and necessary option to alleviate this fungal toxin.

### BRIEF SUMMARY OF THE INVENTION

It is an objective of the present invention to provide compositions and methods that allow for the degradation of aflatoxin before and/or after harvesting, as specified in the independent claims. Embodiments of the invention are given in the dependent claims. Embodiments of the present invention can be freely combined with each other if they are not mutually exclusive.

The present intention features methods for pre-harvest as well as a post-harvest aflatoxin elimination by adding an aflatoxin-degrading enzyme active in dry kernels.

The present invention features an expression cassette. In some embodiments of the expression cassette comprises a gene for an aflatoxin-degrading enzyme and a selectable marker, both operatively linked to a plant-specific promoter. In some embodiments, the expression cassette comprises a plant-specific promoter operatively linked to a endoplasmic reticulum (ER) signal sequence, a gene for an aflatoxin-degrading enzyme operatively linked to the ER-signal sequence, and an ER retention sequence operatively connected to the aflatoxin-degrading enzyme. In further embodiments, the expression cassette comprises a plant-specific promoter operatively linked to a endoplasmic reticulum (ER) signal sequence, a gene for an aflatoxin-degrading enzyme operatively linked to the ER-signal sequence, an ER retention sequence operatively connected to the aflatoxin-degrading enzyme, and a selectable marker operatively linked to the ER-retention sequence.

The present invention may also feature a transgenic plant engineered to degrade Aspergillus aflatoxin. In some embodiments, the transgenic plant expresses an expression cassette comprising a gene for an aflatoxin-degrading enzyme and a selectable marker both operatively linked to a plant-specific promoter. In other embodiments, the transgenic plant expresses an expression cassette comprising a plant-specific promoter operatively linked to a endoplasmic reticulum (ER) signal sequence, a gene for an aflatoxin-degrading enzyme operatively linked to the ER-signal sequence, and an ER retention sequence operatively connected to the aflatoxin-degrading enzyme. In further embodiments, the transgenic plant expresses an expression cassette comprising a plant-specific promoter operatively linked to a endoplasmic reticulum (ER) signal sequence, a gene for an aflatoxin-degrading enzyme operatively linked to the ER-signal sequence, an ER retention sequence operatively connected to the aflatoxin-degrading enzyme, and a selectable marker operatively linked to the ER-retention sequence.

The present invention further features a method of producing a transgenic plant capable of degrading aflatoxin after harvesting. In some embodiments, the method comprises introducing an expression cassette into a plant cell. In some embodiments, the expression cassette comprises a plant-specific promoter operatively linked to a endoplasmic reticulum (ER) signal sequence, a gene for an aflatoxin-degrading enzyme operatively linked to the ER-signal sequence, an ER retention sequence operatively connected to the aflatoxin-degrading enzyme, and a selectable marker operatively linked to the ER-retention sequence. In other embodiments, the method comprises regenerative the plant cell to produce a plant such that the plant degrades aflatoxin.

One of the unique and inventive technical features of the present invention is the expression of the aflatoxin-degrading enzyme gene in a transgenic plant. Without wishing to limit the invention to any theory or mechanism, it is believed that the technical feature of the present invention advantageously provides for the degradation of toxins pre- and post-harvest. None of the presently known prior references or work has the unique inventive technical feature of the present invention.

Furthermore, the inventive technical features of the present invention contributed to a surprising result. For example, the present invention features a method of expressing a toxin-degrading enzyme in edible plant tissue with subcellular targeting of the enzyme to enhance the stability of the enzyme and allow for large accumulation of the enzyme.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic of an embryo-specific ER-targeted aflatoxin degrading enzyme expression cassette used to transform a plant (e.g., maize). An embryo-specific promoter was used to drive the expression of a codon-optimized fungal aflatoxin-degrading enzyme. ER targeting sequences were added to both the 5′ (SP, signal peptide) and 3′ (KHDEL) of the open reading frame. Using this expression cassette transgenic maize plants can be generated. NOS=Nopaline synthase gene. The bottom schematic shows a synthetic construct comprising a section of the embryo-specific Zea mays globulin 1 promoter driving expression of an ER-targeted plant codon optimized 2.166 kb open reading frame encoding for Armillariella tabescenes' aflatoxin-degrading enzyme. ER-targeting elements were added and consisted of a 5′ addition of a 22 amino-acid encoding signal sequence from the Arabidopsis chitinase gene and the 3′ addition of the ER retention KHDEL motif.

FIGS. 2A-2D show an ER-targeted GFP in transgenic seed. FIG. 2A shows white light and FIG. 2B shows blue light expressing ER-targeted/retained GFP (left) compared to a non-transgenic seed (right). ER-targeted GFP is accumulated in stable protein bodies as shown in FIG. 2C fluorescent light micrograph. FIG. 2D shows a transmission electron immunoassay using anti-GFP gold labeled antibodies.

FIG. 3 shows regeneration of transgenic maize. Agrobacterium-transformed callus on shoot-induction media selecting for the Bar gene.

FIG. 4 shows aflatoxin quantification in transgenic maize kernels. Toxin-producing Aspergillus flavus was injected into 20 DAP maize cobs and allowed to grow for 14 days. Kernels surrounding each infection site were harvested and assayed for toxin. Aflatoxin levels (log scale) in biological replicates of the transgenic ENZ7, ENZ8, and ENZ10 and 3 non transgenic controls (WT1, WT2, WT3) cobs as determined by TLC analysis.

FIGS. 5A-5B show Aspergillus flavus infection and aflatoxin quantification in Enz transgenic maize. Freshly grown spore suspensions of A. flavus AF13 were injected into maize developing cobs and allowed to infect kernels. Shown FIG. 5A are two representative infection sites with husk intact (left) and husk removed (right) immediately prior to kernel harvest for aflatoxin quantification. Infected cobs were harvested at either 14-days or 30-days post-infection. Cobs had 4 infection sites each with up to 2 biological replicates for nulls and 4-5 biological replicates for each of the 3 transgenic lines (Enz7, Enz8, and Enz10). FIG. 5B shows total aflatoxins were extracted from harvested kernels surrounding each infection site and quantified by thin layer chromatography followed by scanning densitometry. Shown for each sample is the average log ppb±SE, nd denotes undetectable at a detection limit of 20 ppb. Averages of all three Enz transgenic lines were determined to be significantly different (denoted by *) from the nontransgenic null at both 14- and 30-day infection treatments as determined by student tests p<0.05

FIG. 6 shows dry kernel infection assay of ENZ transgenic maize. * denotes means are significantly different from null nontransgenic using 3 replicates and student t-test p<0.05.

FIG. 7 shows dry kernel infection assay of ENZ transgenic maize. Pollen counts shown as means+/−SE of three replicates. * denotes means are significantly different from null nontransgenic as determined by student t test p<0.05.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, the disclosures of which are incorporated in their entirety by reference herein

All publications, patent applications, patents, and other references mentioned herein are incorporated by reference in their entirety. Although methods and materials similar or equivalent to those described herein can be used to practice or test the disclosed technology, suitable methods and materials are described below. The materials, methods, and examples are illustrative only and not intended to be limiting.

The term “vector or construct” may refer to any nucleic acid that acts as a carrier for other (e.g., foreign) nucleic acid sequences that are not native to the vector. When introduced into an appropriate host cell, a vector may replicate itself (and, thereby, the foreign nucleic acid sequence) or express at least a portion of the foreign nucleic acid sequence. In one context, a vector is a linear or circular nucleic acid into which a nucleic acid sequence of interest is introduced (for example, cloned) for the purpose of replication (e.g., production) and/or manipulation using standard recombinant nucleic acid techniques (e.g., restriction digestion). A vector can include nucleic acid sequences that permit it to replicate in a host cell, such as an origin of replication. A vector can also include one or more selectable marker genes and other genetic elements known in the art. Common vectors include, for example, plasmids, cosmids, phage, phagemids, artificial chromosomes (e.g., BAC, PAC, HAC, YAC), and hybrids that incorporate features of more than one of these types of vectors. Typically, a vector includes one or more unique restriction sites (and in some cases a multi-cloning site) to facilitate insertion of a target nucleic acid sequence.

The term “transgenic plant” may refer to any plant whose DNA has been modified using genetic engineering techniques, well known in the art, to introduce a new trait to the plant which does not occur naturally in the species.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Aflatoxin is produced by certain fungal species as an energy source only they themselves can consume. Screening revealed a number of microorganism sources that degrade aflatoxin via an enzymatic reaction to non-toxic compounds. An aflatoxin-degrading enzyme has been characterized and it is encoded by a gene cloned from honey mushrooms (Armillariella tabescens; genbank accession AY941095). Honey mushrooms are nontoxic and edible. This mushroom produces an enzyme that has been identified and characterized to degrade the aflatoxin B1 molecule, the most common and toxic of the aflatoxins. The enzyme encoding gene has been cloned and it is a 2 kb gene encoding for a 695 amino acid protein. This protein sequence was searched via the World Health Organization (WHO) decision tree ranks to determine potential allergenicity and results deemed the protein to be very unlikely to be allergenic as it displays less than 1% homology to any known allergen. The open reading from this fungal gene was codon-optimized (Table 1) for expression in maize seeds using a codon-optimization table.

TABLE 1

| Description | Sequence: |
|---|---|
| SEQ ID NO: 1 | MATTTVHRERFLADKSAPLCGMDIRKSFDQLSSKEKLYTHYVTEAS |
| Codon-optimized | WAGARIIQAQWTPQATDLYDLLILTFSVNGKLADLNALKTSSGLSED |
| aflatoxin-degrading | DWEALIQYTVQVLSNLVNYKTFGFTKIIPRVDAEKFESVVKASSNAD |
| enzyme from Armillaria | QGSALFTKLKQHIYALSPESALFIGKRKDGHVSNYYLGEPVGDAEV |
| tabescens | DAIQNVAEKLGVDILNTRVKKNGAGDYTLLVASAKTSPPSVHDFQID |
| | STPAKLTIEYGDYASSLTKVVAALQEAKQYTANDHQSAMIEGYVKSF |
| | NSGSIPEHKAASTEWVKDIGPVVESYIGFVETYVDPYGGRAEWEGF |
| | TAIVDKQLSAKYEALVNGAPKLIKSLPWGTDFEVDVFRKPDFTALEV |
| | VSFATGGIPAGINIPNYYEVRESTGFKNVSLANILAAKVPNEELTFIHP |
| | DDVELYNAWDSRAFELQVANHELLGHGSGKLFQEGADGKLNFDPE |
| | KVINPLTGKPITSWYKPGQTPDSVLGEVSSSMEECRAETVALYLVSN |
| | LDILKIFNYVDKQDIEDIQYITFLLMARAGLRALEFYDPATKKHGQAH |
| | MQARMGITQYLIQAGIARLELIQDANGELENLYVRVDREKVLSKGKE |
| | VVGQLLIELQVRKSTADGTGSRDFYTTLTEPISGWEGKIRDIVLKKKL |
| | PRKIFVQPNTFVVNGEVQLKEYPLTAAGVIESFIERRL |

The stability of introduced proteins is typically an issue in dry seeds. An introduced protein without any subcellular targeting is often accumulated in very small quantities or is degraded as seeds dry. Therefore, the present invention features compositions and methods for subcellular targeting of proteins for stable accumulation using the method of subcellular targeting to the endoplasmic reticulum (ER). In order to target an introduced protein to the ER there are two additions to an open reading frame of a gene; (1) a signal peptide is added to the 5′ end of the gene, and (2) an ER retention tag is added to the 3′ end of the open reading frame. These two elements have been shown to be effective at both targeting and retaining an introduced protein to the ER and allow for its stable accumulation in dry seeds.

Without wishing to limit the present invention to any theories or mechanisms it is believed that targeting the introduced aflatoxin-degrading enzyme to the ER subcellular compartment in maize cells should enable both stable accumulation and expression in dry maize kernels and thereby add in the elimination of post-harvested production of aflatoxin.

The present invention features expression cassette comprising an aflatoxin-degrading enzyme targeted to the ER by placing at the 5′ end in-frame to the 2,000 bp open reading frame of the enzyme a 20-amino acid ER signal sequence from the Arabidopsis chitinase gene and the nucleotides encoding for the known ER retention KHDEL sequence at the 3′ end of the open reading frame immediately in-front of stop codons.

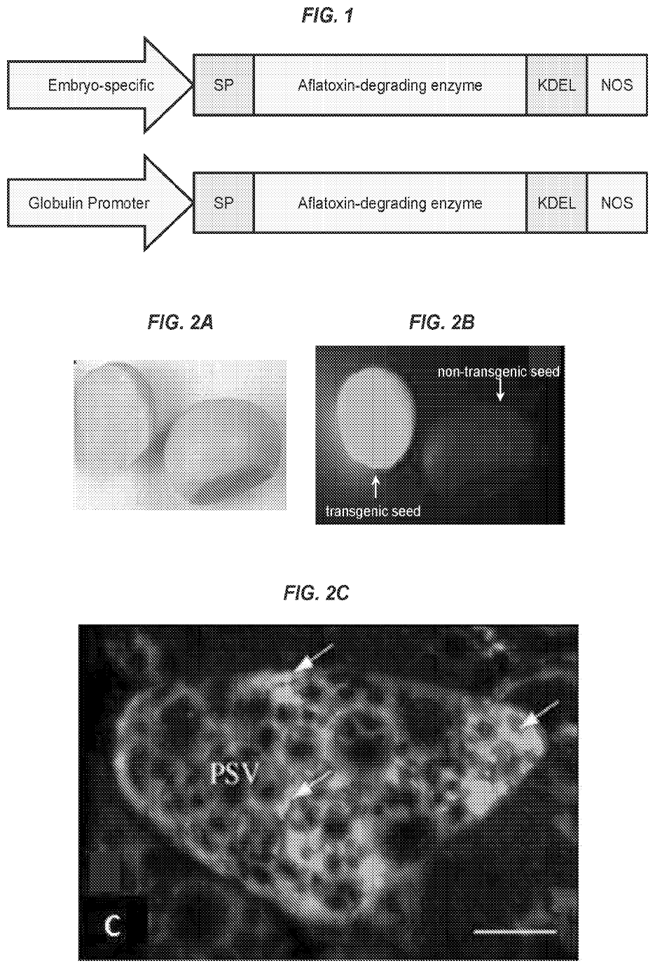

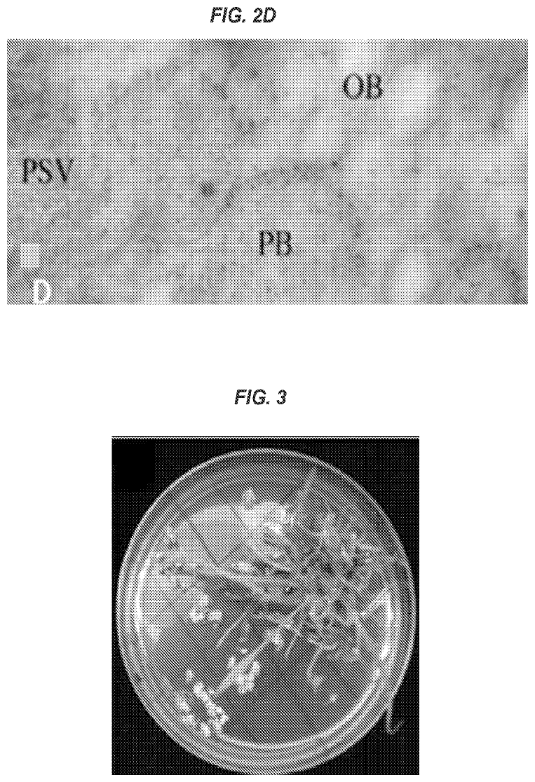

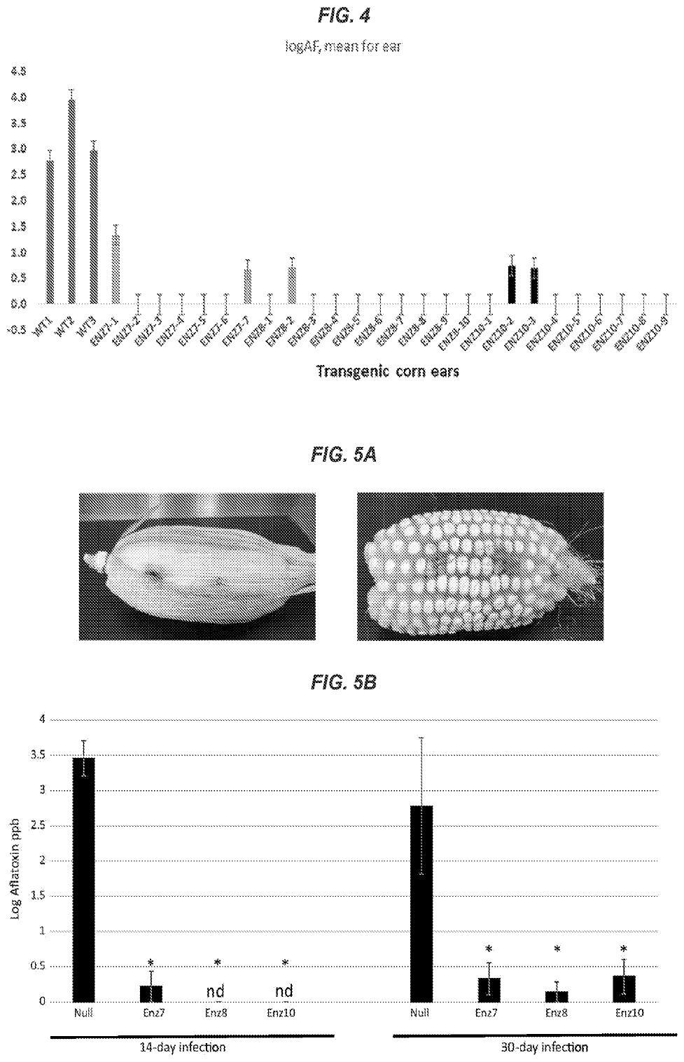

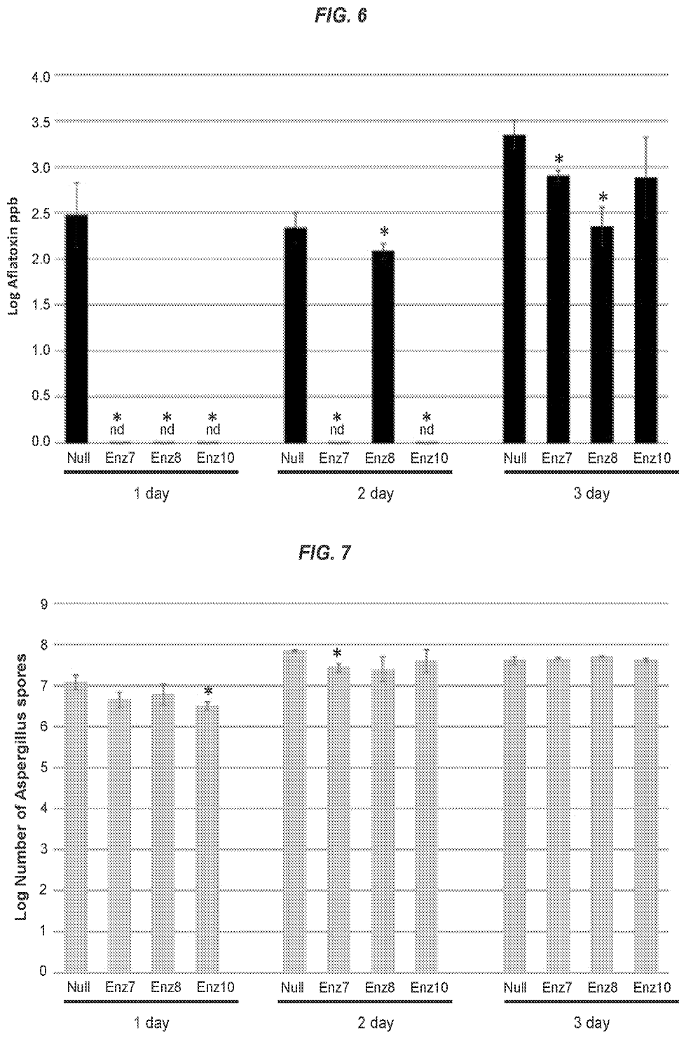

Referring now to FIGS. 1-7, the present invention features compositions and methods for degrading aflatoxin pre- and post-harvest.

The present invention features an expression cassette. The expression cassette may comprise a gene for an aflatoxin-degrading enzyme and a selectable marker both operatively linked to a plant-specific promoter. In some embodiments, the expression cassette comprises a plant-specific promoter operatively linked to a endoplasmic reticulum (ER) signal sequence, a gene for an aflatoxin-degrading enzyme operatively linked to the ER-signal sequence, and a selectable marker operatively linked to the aflatoxin-degrading enzyme. In other embodiments, the expression cassette comprises a plant-specific promoter operatively linked to a endoplasmic reticulum (ER) signal sequence, a gene for an aflatoxin-degrading enzyme operatively linked to the ER-signal sequence, an ER retention sequence operatively connected to the aflatoxin-degrading enzyme, and a selectable marker operatively linked to the ER-retention sequence.

In some embodiments, the expression cassette comprises a plant-specific promoter operatively linked to a endoplasmic reticulum (ER) signal sequence, and a gene for an aflatoxin-degrading enzyme operatively linked to the ER-signal sequence. In other embodiments, the expression cassette comprises a plant-specific promoter operatively linked to a endoplasmic reticulum (ER) signal sequence, a gene for an aflatoxin-degrading enzyme operatively linked to the ER-signal sequence, and an ER retention sequence operatively connected to the aflatoxin-degrading enzyme.

TABLE 2

| Description | Sequence |
|---|---|
| SEQ ID NO: 2 | MKTNLFLFLIFSLLLSLSSAEFMATTTVHRERFLADKSAPLCGMDI |
| protein encoding section | RKSFDQLSSKEKLYTHYVTEASWAGARIIQAQWTPQATDLYDLLIL |
| of expression cassette | TFSVNGKLADLNALKTSSGLSEDDWEALIQYTVQVLSNLVNYKTF |
| with 5″ ER signal and 3′ | GFTKIIPRVDAEKFESVVKASSNADQGSALFTKLKQHIYALSPESA |
| ER retention underlined | LFIGKRKDGHVSNYYLGEPVGDAEVDAIQNVAEKLGVDILNTRVK |
| | KNGAGDYTLLVASAKTSPPSVHDFQIDSTPAKLTIEYGDYASSLTK |
| | WVAALQEAKQYTANDHQSAMIEGYVKSFNSGSIPEHKAASTEWV |
| | KDIGPVVESYIGFVETYVDPYGGRAEWEGFTAIVDKQLSAKYEAL |
| | VNGAPKLIKSLPWGTDFEVDVFRKPDFTALEVVSFATGGIPAGINI |
| | PNYYEVRESTGFKNVSLANILAAKVPNEELTFIHPDDVELYNAWD |
| | SRAFELQVANHELLGHGSGKLFQEGADGKLNFDPEKVINPLTGKP |
| | ITSWYKPGQTPDSVLGEVSSSMEECRAETVALYLVSNLDILKIFNY |
| | VDKQDIEDIQYITFLLMARAGLRALEFYDPATKKHGQAHMQARMG |
| | ITQYLIQAGIARLELIQDANGELENLYVRVDREKVLSKGKEVVGQL |
| | LIELQVRKSTADGTGSRDFYTTLTEPISGWEGKIRDIVLKKKLPRKI |
| | FVQPNTFVVNGEVQLKEYPLTAAGVIESFIERRLKHDEL |

The present invention also features a transgenic plant engineered to degrade Aspergillus aflatoxin. In some embodiments, the transgenic plant expresses an expression cassette comprising a gene for an aflatoxin-degrading enzyme and a selectable marker both operatively linked to a plant-specific promoter. In other embodiments, the transgenic plant expresses an expression cassette comprising a plant-specific promoter operatively linked to a endoplasmic reticulum (ER) signal sequence, a gene for an aflatoxin-degrading enzyme operatively linked to the ER-signal sequence, and a selectable marker operatively linked to the aflatoxin-degrading enzyme. In further embodiments, the transgenic plant expresses an expression cassette comprising a plant-specific promoter operatively linked to a endoplasmic reticulum (ER) signal sequence, a gene for an aflatoxin-degrading enzyme operatively linked to the ER-signal sequence, an ER retention sequence operatively connected to the aflatoxin-degrading enzyme, and a selectable marker operatively linked to the ER-retention sequence.

In some embodiments, the transgenic plant expresses an expression cassette comprising a plant-specific promoter operatively linked to a endoplasmic reticulum (ER) signal sequence, and a gene for an aflatoxin-degrading enzyme operatively linked to the ER-signal sequence. In other embodiments, the transgenic plant expresses an expression cassette comprising a plant-specific promoter operatively linked to a endoplasmic reticulum (ER) signal sequence, a gene for an aflatoxin-degrading enzyme operatively linked to the ER-signal sequence, and an ER retention sequence operatively connected to the aflatoxin-degrading enzyme. In some embodiments, the present invention features a transgenic plant comprising an expression cassette described herein.

In some embodiments, the expression cassette further comprises an ER-signal sequence operatively linked to the 5′ end of the gene for an aflatoxin-degrading enzyme. In other embodiments, the expression cassette further comprises an ER-retention signal operatively linked to the 3′ end of the gene for an aflatoxin-degrading enzyme.

In some embodiments, the aflatoxin-degrading enzyme is targeted to the ER. In other embodiments, the aflatoxin-degrading enzyme is targeted to the ER via an ER-signal sequence. In some embodiments, the ER-signal sequence is operatively linked to the 5′ end of the gene for an aflatoxin-degrading enzyme. In other embodiments, the ER-signal sequence is from the Arabidopsis chitinase gene. In further embodiments, any ER-signal sequence well known in the art may be used to target the aflatoxin-degrading enzyme protein to the endoplasmic reticulum (ER).

In some embodiments, a “ER-signal sequence” may refer to a short hydrophobic peptide (usually 16-30 amino acids long) present at the N-terminus of the majority of newly synthesized proteins that are destined toward the secretory pathway (specifically the endoplasmic reticulum).

In some embodiments, the aflatoxin-degrading enzyme is retained in the ER. In some embodiments, the aflatoxin-degrading enzyme is stored in the ER. In other embodiments, the aflatoxin-degrading enzyme is retained (i.e. stored) in the ER via a KHDEL sequence (SEQ ID NO: 3) or a KDEL sequence (SEQ ID NO: 4). In some embodiments, the ER-retention sequence is operatively linked to the 3′ end of the gene for an aflatoxin-degrading enzyme. In further embodiments, the aflatoxin-degrading enzyme is stored in the ER. In further embodiments, any ER-retention sequence well known in the art may be used to retain the aflatoxin-degrading enzyme protein to the endoplasmic reticulum (ER).

In some embodiments, proteins described herein comprise a 5′ ER-signal sequence coupled with a 3′ KHDEL sequence, which allows for both the targeting and retention of the protein in the endoplasmic reticulum (ER).

In some embodiments, the aflatoxin-degrading enzyme is from Armillariella tabescens. In other embodiments, the aflatoxin-degrading enzyme degrades aflatoxin B1 molecules. In further embodiments, the aflatoxin-degrading enzyme is from Aspergillus. In other embodiments, the aflatoxin-degrading enzyme is from a fungal species that interacts with Aspergillus.

In some embodiments, the plant-specific promoter is an embryo specific promoter. Without wishing to limit the present invention to any theories or mechanisms it is believed that the embryo-specific promoter functions in dry post-harvest kernels.

In some embodiments, the plant specific promoter is a globulin-1 promoter. In other embodiments, the plant specific promoter is an endosperm promoter. In some embodiments, the plant specific promoter is a glycinin promoter. In some embodiments, the plant specific promoter is a plant pathogen promoter. In further embodiments, the plant specific promoter drives expression in the embryo. In some embodiments, any promoter that allows for protein expression within an edible portion of a crop may be used in the present invention.

Without wishing to limit the present invention to any theories or mechanisms it is believed that a plant pathogen promoter (e.g., a fungal-infection responsive promoter) may drive the aflatoxin-degrading enzyme production so the degradation of the aflatoxin produced can keep pace with the production of the toxin while the Aspergillus infection progresses.

In some embodiments, the aflatoxin-degrading gene is expressed in the embryo. In other embodiments, the aflatoxin-degrading gene is expressed in the aleurone tissue.

In some embodiments, the expression cassette may comprise any plant-specific promoter that directs expression to the edible portion of a plant can be operatively linked to any endoplasmic reticulum (ER) signal sequence, any gene for an aflatoxin-degrading enzyme operatively linked to a ER-signal sequence, and any ER retention sequence operatively connected to a aflatoxin-degrading enzyme.

In some embodiments, the selectable marker comprises a bialaphos resistance (bar) gene. In some embodiments, the selectable marker comprises an antibiotic resistant marker. In other embodiments, other selectable markers well known in the art, plant biotechnology, may be used including but not limited to hygromycin resistance or kanamycin resistance markers.

In some embodiments, the transgenic plant is a maize species or a peanut species. In other embodiments, the transgenic plant is any crop which aflatoxin infects. Non-limiting examples include, but are not limited to, rice, sorghum, soybean, cassava, millet or cotton.

In some embodiments, the transgenic plant is engineered to degrade Aspergillus aflatoxin after the transgenic plant is harvested (i.e., post-harvest). In other embodiments, the transgenic plant is engineered to degrade Aspergillus aflatoxin before the transgenic plant is harvested (i.e., pre-harvest). In further embodiments, the transgenic plant is engineered to degrade Aspergillus aflatoxin before and after the transgenic plant is harvested.

In further embodiments, the present invention also features a method of producing a transgenic plant capable of degrading aflatoxin after harvesting. In some embodiments, the method comprises introducing an expression cassette into a plant cell. In other embodiments, the expression cassette comprises a plant-specific promoter operatively linked to a endoplasmic reticulum (ER) signal sequence, a gene for an aflatoxin-degrading enzyme operatively linked to the ER-signal sequence, an ER retention sequence operatively connected to the aflatoxin-degrading enzyme, and a selectable marker operatively linked to the ER-retention sequence. In other embodiments, the method comprises regenerating the plant cells to produce a plant such that the plant is able to cause the degradation of aflatoxin.

### Example

The following is a non-limiting example of the present invention. It is to be understood that said example is not intended to limit the present invention in any way. Equivalents or substitutes are within the scope of the present invention.

Regenerated transgenic maize plants are self-pollinated and 1 plant/line harvested for kernels for expression analysis. Total RNA is extracted from developing maize kernels and used to produce cDNA for quantitative real-time expression (RT-PCR) analysis using primers specific to the inserted aflatoxin enzyme sequence and using an endogenous constitutive actin gene as a control. Confirmed transgene-expressing maize plants are self-pollinated and planted for two generations, each 4-5 months in duration. Leaf painting assays are used to determine the expression of the bialaphos resistance selectable marker gene on 20 plants/line to determine when each line is homozygous for the inserted transgenes. For painting assays, an approximately 3.8 cm adaxial tip surface of a fully expanded leaf of ˜ 10 day old plant will be sprayed with a 3 mg/ml glufosinate ammonium solution with the treated area marked. Resistance will be visible and scored 7 days after treatment. Lines that are expressing the inserted aflatoxin enzyme and shown to reach homozygosity will be further assayed by molecular analysis using a PCR based technique pioneered by the inventor to confirm homozygosity.

Aflatoxin-degradation expression cassette: The aflatoxin-degrading enzyme from the Honey fungus Armillariella tabescens (GenbankAccession AY941095) comprising a 2166 bp open-reading frame with both ER-signal and ER-retention tags flanking the aflatoxin-degrading encoding a 695 amino acid protein was synthesized (Celtek Genes) using a plant codon optimization table. This enzyme's open reading frame was placed in-frame between elements to subcellularly localize the protein to the ER by the addition of the 22 amino-acid ER signal sequence from the Arabidopsis chitinase gene at the 5′ end and the ER retention KHDEL motif at the 3′ end of the open reading frame (FIG. 1). The ER-targeted enzyme-encoding gene was then placed under the direction of an embryo-specific promoter. A 1.4 kb region of the Zea mays globulin-1 promoter (Genbank Accession AH001354.2) was synthetically manufactured and used in the expression cassette for the embryo-specific expression of an aflatoxin-degrading enzyme in maize. The embryo-directed expression of the aflatoxin-degrading enzyme cassette was subsequently cloned into an Agrobacterium tumefaciens plasmid pTF1010.1 that contains the constitutively expressed selectable marker bar resistance gene (phosphinothricin acetyltransferase). The resultant cassette was hereafter referred to as glob::Enz.

Transgenic maize production: Transgenic maize (Zea mays Hi II hybrid A 188 and B73 background) expressing the glob::Enz cassette were produced using Agrobacterium-mediated transformation protocol. Plantlets from ten putative transgenic lines were obtained after tissue-culture selection on media containing the selectable agent, bialophos. Each transgenic line was confirmed by PCR to be containing the glob::Enz cassette by genomic PCR using primers specific to the cassette (Enz-For 5′-GTTGGCAGATCTTAACGCTCT-3′ (SEQ ID NO: 5), Enz-Rev 5′-CTTCCCATTCAGCCCTACCTC-3′ (SEQ ID NO: 6)) producing an expected amplicon of 743 bp). Standard PCR conditions were used in the PCR screening: 50 ng genomic DNA, 1×Taq DNA polymerase buffer, 2U Taq polymerase (New England Biolabs), 250 UM of each dNTP and 200 nM of each primer with PCR conditions of an initial denaturation (94° C., 4 min) and 45 amplification cycles (94° C., 30 s; 55° C., 30 s; 72° C. 60 s) followed by a final elongation step (72° C., 7 m). Transgenic lines were grown to the T3 generation by repetitive self-pollination and ensured stable transmission of the Enz cassette by performing Enz-specific PCR reaction screening on progeny each generation.

Transgene expression in transgenic maize kernels: Transgenic glob::Enz transgenic maize lines were screened by expression of the inserted cassette of interest by detecting the Enz transcript in developing kernels. RNA was extracted using RNeasy Kit (Qiagen) by grinding approximately 10 DAP kernels in liquid nitrogen harvested from three stable transgenic lines (Enz-7, Enz-8, Enz-10) and nontransgenic (null) control kernels. First-stand cDNA synthesis was performed using 1 μg of total RNA per sample, 9 μl of 2 M betaine monohydrate (Sigma) and random primers using RevertAid First Strand cDNA Synthesis Kit (Thermo Scientific) according to the manufacturer's instructions. Standard PCR conditions were as described in transgenic maize screening above using primers specific to the Enz open reading frame (as above) and primers specific to the control maize actin gene. The maize actin primers were designed adjacent to an intron, so there was an expected amplicon size differential if genomic DNA or cDNA was amplified, 264 bp and 157 bp, respectively. PCR amplicon products were separated on a 1% (w/v) agarose gel (Sigma-Aldrich) mixed with ethidium bromide (0.5 μg/ml) (Sigma-Aldrich) using a 100 pb DNA Ladder (Fisher Scientific) and mass subsequently imaged under ultraviolet light.

Once the transgenic maize lines have been characterized for the insertion and expression of the toxin-degrading enzyme encoding gene cassette, then Aspergillus challenges will be conducted in both pre-harvest and post-harvest conditions. At least three technical replicates for at least three biological transgenic lines are used in the challenges along with a nontransgenic counterpart controls. The pre-harvest conditions are performed using the known toxin-producing A. flavus AF13 strain and the infections are allowed to occur for 2 weeks and 1 month durations. After infections, all live kernels surrounding each infection site are harvested and quantified by TLC plate analysis for toxin loads.

Similarly, for post-harvest conditions, whole dry kernels from the transgenic maize plants are infected with A. flavus AF13 strain and then tested for the presence and amount of aflatoxin production. At least 20 intact kernels harvested from greenhouse grown transgenic maize plants from each of 3 chosen enzymes expressing transgenic lines are fungal challenged. Kernels from each maize transgenic line have at least 5 technical replicates. Treatments are arranged in a randomized complete block design. For both the toxin results obtained from pre- and post-harvest experiments, statistics are performed to determine if there are differences in the toxin accumulated in the transgenic lines versus the controls.

To both confirm and quantitate the presence of the aflatoxin enzyme mass spectroscopy analysis is preformed on the dry transgenic maize kernels. The three transgenic lines determined by RT-PCR to be expressed will be the lines used in Aspergillus-challenges as well as analyzed for the total proteome of the dry kernels. The decision to not add an epitope tag, such as FLAG, to the open reading frame of the toxin-degrading enzyme was made to ensure the correct folding and function of the enzyme. A correlation of the amount of enzyme produced per transgenic event and the level of toxin present in the kernels will be determined as an indication of how effective the toxin-degrading enzyme will be in both pre- and post-harvest situations.

Aspergillus flavus culture propagation: Aspergillus flavus isolate AF13 from the USDA-ARS Aflatoxin Biocontrol Lab culture collection was grown from long-term silica gel stocks by placing a single silica granule on the center of 5/2 agar (5% V-8 vegetable juice and 2% agar, pH 5.2) and incubating the plate in the dark at 31° C. for 5 days. Agar plugs (7-10 per vial) were transferred to water vials which containing 3.5 ml of ddH2O. Spore suspensions (15 μl) from water vial stocks were seeded in the center well of 5/2 agar plates, and after incubation at 31° C. for 5-7 days, spores were picked up from plates using sterile cotton swabs and suspended in 10 ml of sterile 0.02% Tween-80. Spore suspensions were vortexed, and 1.2 ml of the suspension was added to 10.8 ml of 50% ethanol. Turbidity in nephelometric turbidity units (NTUs) was measured using a calibrated turbidimeter (Orbeco-Hellige Farmingdale NY, model 965-10). The final spore concentration was calculated using a standard curve for NTU versus spores/ml using the formula: spores/ml=NTU×49,937. The spore suspension was then diluted to a final concentration of 1.0×107 spores/ml in sterile distilled water.

Aspergillus flavus infection assays and aflatoxin quantificaion: At 8 to 10 days after pollination (DAP), ears on transgenic maize plants and nontransgenic null control plants grown side-by-side under greenhouse conditions were wounded at four spots by pushing a 3-mm diameter cork-borer through the husk to a depth of approximately 5 mm. Each wound was inoculated with 10 μl of the A. flavus conidial suspension. In each experiment, 3 to 5 ears of each transgenic line (Enz 7, Enz 8, and Enz 10) and at least one non-transgenic null ear were inoculated. After 30 and 14 days in the first and second experiment, respectively, ears were harvested and dried at 45° C. for 3 to 4 days. Eight to nine kernels surrounding each inoculated wound were removed from the ears, weighed, and ground. For each sample, total aflatoxins (aflatoxin B1+aflatoxin B2) were extracted from 1.5 g ground kernels with 15 ml of 70% methanol, and extracts were separated using thin layer chromatography (TLC) and aflatoxin was quantified using scanning densitometry as described previously [59]. Briefly, 12 μl of extract was spotted on 20×20-cm TLC glass plates (Silica Gel 60 F254, Millipore) along with an aflatoxin standard (Aflatoxin Mix Kit-M, Supelco, Bellefonte, PA) and plates were developed with diethyl ether:methanol:water (96:3:1). The presence or absence of aflatoxins B1 and AB2 were confirmed visually under ultraviolet light (365 nm) and quantified on plates using scanning fluorescence densitometry with a CAMAG TLC Scanner 3 (Camag Scientific Inc.). Quantities of aflatoxin relative to the standard were used to calculate total ng aflatoxin per g kernels (parts per billion; ppb). Values are presented as average log ppb±standard error and determined to be significant at p<0.05 by performing student t-tests comparing each transgenic event to the nontransgenic control.

Reducing aflatoxin in maize kernels: The three transgenic Enz maize lines characterized for the insertion and expression of the aflatoxin-degrading enzyme encoding gene cassette were inoculated with A. flavus in pre-harvest conditions. At least three technical replicates for the three stable biological transgenic Enz lines were used in the challenges along with nontransgenic (null) counterpart controls. Multiple infection sites were made into developing cobs of biological replicate plants of the 3 Enz transgenic lines (Enz7, Enz8 and Enz10) along with side-by-side greenhouse grown nontransgenic null plants by inoculating 8-10 days after pollination (DAP) cobs with 10 μl of a freshly grown A. flavus AF13 spore suspension (1.0×107 spores/ml) in sterile distilled water (FIG. 5A). The infections were allowed to progress for 14 or 30 days duration. After infections, all live kernels surrounding each infection site were harvested and total aflatoxins were extracted, separated by thin layer chromatography (TLC), and quantified on TLC plates using scanning densitometry. FIG. 5B shows aflatoxin concentrations after developing kernels were infected with A. flavus and the infection was allowed to occur for 14 days. Compared to null nontransgenic maize developing kernels, the three aflatoxin-degrading enzyme expressing transgenic lines (Enz7, Enz8 and Enz10) had significantly reduced aflatoxin concentrations (student t-test p<0.05) with 2 of the lines having undetectable levels of aflatoxin (<20 ppb). As shown in FIG. 5B, after a 14-infection day duration, nontransgenic null maize kernels contained 3.46±0.25 ppb log aflatoxin compared to 0.22±0.22 ppb in transgenic line Enz 7 and non-detectable levels in both Enz8 and Enz10 transgenic lines. The TLC methodology employed has a detection limit of 20 ppb (log value 1.30 ppb) and given that 20 ppb is the tightest aflatoxin limit in the US for food items destined for direct human consumption, this aflatoxin-degrading enzyme method of the reduction or elimination of this carcinogenic compound from maize, or similarly Aspergillus-infected crops, is very feasible as this strategy should play a significant role towards eliminating crop losses due to this fungal contaminant.

Likewise, FIG. 5B shows aflatoxin loads from similarly infected developing kernels of transgenic Enz maize where the A. flavus infection was allowed to proceed for 1 month before harvest and aflatoxin quantification. Again, as with the 14-day infection period, all 3 expressing aflatoxin-degrading enzyme transgenic maize lines displayed significantly reduced aflatoxin loads compared to the nontransgenic null kernels. Although there was substantial variation, the transgenic lines accumulated at least a 90-fold reduction in aflatoxin after a 30-day A. flavus infection period with null kernels having an average 2.78±0.97 ppb log aflatoxin with Enz 7 accumulating 0.33±0.22 ppb, Enz 8 accumulating 0.14±0.14 ppb and Enz 10 accumulating 0.36±0.29 ppb. All aflatoxin accumulated in the three Enz transgenic lines was determined to be significantly reduced from the nontransgenic null controls by student tests p<0.05. Even with a 30-day infection period, the embryo-expressed aflatoxin-degrading enzyme was able to convert the carcinogenic aflatoxin produced by the contaminating A. flavus fungus to substantially reduced levels in all three transgenic maize lines.

### Embodiments

The following embodiments are intended to be illustrative only and not to be limiting in any way.

Embodiment 1: An expression cassette comprising a gene for an aflatoxin-degrading enzyme and a selectable marker, both operatively linked to a plant-specific promoter.

Embodiment 2: The expression cassette of embodiment 1 further comprising an ER-signal sequence operatively linked to the 5′ end of the gene for an aflatoxin-degrading enzyme.

Embodiment 3: The expression cassette of embodiment 2, wherein the ER-signal sequence is from an Arabidopsis chitinase gene.

Embodiment 4: The expression cassette of embodiment 1, further comprising an ER-retention signal operatively linked to the 3′ end of the gene for an aflatoxin-degrading enzyme.

Embodiment 5: The expression cassette of embodiment 4, wherein the ER-retention signal comprises the sequence KHDEL.

Embodiment 6: The expression cassette of any of embodiments 1-5, wherein the aflatoxin-degrading enzyme is targeted in the ER.

Embodiment 7: The expression cassette of any of embodiments 1-6, wherein the aflatoxin-degrading enzyme is stored in the ER.

Embodiment 8: The expression cassette of embodiment 1, wherein the aflatoxin-degrading enzyme is from Armillariella tabescens.

Embodiment 9: The expression cassette of embodiment 1 or 8, wherein the aflatoxin-degrading enzyme degrades aflatoxin B1 molecules.

Embodiment 10: The expression cassette of embodiment 1, wherein the plant-specific promoter comprises an endosperm promoter or a glycinin promoter.

Embodiment 11: The expression cassette of embodiment 1, wherein the selectable marker comprises a bialaphos resistance (bar) gene.

Embodiment 12: A transgenic plant comprising an expression cassette of any of embodiments 1-11.

Embodiment 13: A transgenic plant engineered to degrade Aspergillus aflatoxin, the transgenic plant expressing an expression cassette comprising a gene for an aflatoxin-degrading enzyme and a selectable marker both operatively linked to a plant-specific promoter.

Embodiment 14: The transgenic plant of embodiment 13 further comprising an ER-signal sequence operatively linked to the 5′ end of the gene for an aflatoxin-degrading enzyme.

Embodiment 15: The transgenic plant of embodiment 14, wherein the ER-signal sequence is from the Arabidopsis chitinase gene.

Embodiment 16: The transgenic plant of embodiment 13, further comprising an ER-retention signal operatively linked to the 3′ end of the gene for an aflatoxin-degrading enzyme.

Embodiment 17: The transgenic plant of embodiment 16, wherein the ER-retention signal comprises the sequence KHDEL.

Embodiment 18: The transgenic plant of any of embodiments 13-17, wherein the aflatoxin-degrading enzyme is targeted in the ER.

Embodiment 19: The transgenic plant of any of embodiments 13-18, wherein the aflatoxin-degrading enzyme is stored in the ER.

Embodiment 20: The transgenic plant of embodiment 13, wherein the aflatoxin-degrading enzyme is from Armillariella tabescens.

Embodiment 21: The transgenic plant of embodiment 13 or 20, wherein the aflatoxin-degrading enzyme degrades aflatoxin B1 molecules.

Embodiment 22: The transgenic plant of embodiment 13, wherein the plant-specific promoter comprises an endosperm promoter or a glycinin promoter.

Embodiment 23: The transgenic plant of embodiment 13, wherein the selectable marker comprises a bialaphos resistance (bar) gene.

Embodiment 24: The transgenic plant of embodiment 13, wherein the transgenic plant is a maize species or a peanut species.

Embodiment 25: The transgenic plant of embodiment 13, wherein the transgenic plant is engineered to degrade Aspergillus aflatoxin after the transgenic plant is harvested.

Embodiment 26: A method of producing a transgenic plant capable of degrading aflatoxin after harvesting, the method comprising: (a) introducing an expression cassette into a plant cell, the expression cassette comprising: (i) a plant specific promoter; (ii) a ER-signal sequence operatively linked to the plant specific promoter; (iii) an aflatoxin-degrading enzyme operatively linked to the ER-signal sequence; (iv) an ER-retention sequence operatively linked to the aflatoxin-degrading enzyme; and (v) a selectable marker linked to the ER-retention sequence; and (b) regenerating the plant cell to produce a plant such that the plant degrades aflatoxin.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” Includes embodiments that could be described as “consisting essentially of” or “consisting of”, and as such the written description requirement for claiming one or more embodiments of the present invention using the phrase “consisting essentially of” or “consisting of” is met.

1. An expression cassette comprising a gene for an aflatoxin-degrading enzyme and a selectable marker, both operatively linked to a plant-specific promoter.

2. The expression cassette of claim 1 further comprising an ER-signal sequence operatively linked to the 5′ end of the gene for an aflatoxin-degrading enzyme; wherein the ER-signal sequence is from an Arabidopsis chitinase gene.

3. (canceled)

4. The expression cassette of claim 1, further comprising an ER-retention signal operatively linked to the 3′ end of the gene for an aflatoxin-degrading enzyme; wherein the ER-retention signal comprises the sequence KHDEL.

5. (canceled)

6. The expression cassette of claim 1, wherein the aflatoxin-degrading enzyme is targeted in the ER.

7. The expression cassette of claim 1, wherein the aflatoxin-degrading enzyme is stored in the ER.

8. The expression cassette of claim 1, wherein the aflatoxin-degrading enzyme is from Armillariella tabescens; wherein the aflatoxin-degrading enzyme degrades aflatoxin B1 molecules.

9. (canceled)

10. The expression cassette of claim 1, wherein the plant-specific promoter comprises an endosperm promoter or a glycinin promoter.

11. The expression cassette of claim 1, wherein the selectable marker comprises a bialaphos resistance (bar) gene.

12. (canceled)

13. A transgenic plant engineered to degrade Aspergillus aflatoxin, the transgenic plant expressing an expression cassette comprising a gene for an aflatoxin-degrading enzyme and a selectable marker both operatively linked to a plant-specific promoter.

14. The transgenic plant of claim 13 further comprising an ER-signal sequence operatively linked to the 5′ end of the gene for an aflatoxin-degrading enzyme; wherein the ER-signal sequence is from the Arabidopsis chitinase gene.

15. (canceled)

16. The transgenic plant of claim 13, further comprising an ER-retention signal operatively linked to the 3′ end of the gene for an aflatoxin-degrading enzyme; wherein the ER-retention signal comprises the sequence KHDEL.

17. (canceled)

18. The transgenic plant of claim 13, wherein the aflatoxin-degrading enzyme is targeted in the ER.

19. The transgenic plant of claim 13, wherein the aflatoxin-degrading enzyme is stored in the ER.

20. The transgenic plant of claim 13, wherein the aflatoxin-degrading enzyme is from Armillariella tabescens.

21. The transgenic plant of claim 13, wherein the aflatoxin-degrading enzyme degrades aflatoxin B1 molecules.

22. The transgenic plant of claim 13, wherein the plant-specific promoter comprises an endosperm promoter or a glycinin promoter.

23. The transgenic plant of claim 13, wherein the selectable marker comprises a bialaphos resistance (bar) gene.

24. The transgenic plant of claim 13, wherein the transgenic plant is a maize species or a peanut species.

25. The transgenic plant of claim 13, wherein the transgenic plant is engineered to degrade Aspergillus aflatoxin after the transgenic plant is harvested.

26. A method of producing a transgenic plant capable of degrading aflatoxin after harvesting, the method comprising:
a) introducing an expression cassette into a plant cell, the expression cassette comprising:
i. a plant specific promoter,
ii. a ER-signal sequence operatively linked to the plant specific promoter,
iii. an aflatoxin-degrading enzyme operatively linked to the ER-signal sequence;
iv. an ER-retention sequence operatively linked to the aflatoxin-degrading enzyme; and
v. a selectable marker linked to the ER-retention sequence; and
b) regenerating the plant cell to produce a plant such that the plant degrades aflatoxin.


SEQUENCE LISTING (6 sequences)

SEQ ID NO: 1 - protein, 695 aa, Armillaria tabescens
MATTTVHRERFLADKSAPLCGMDIRKSFDQLSSKEKLYTHYVTEASWAGARIIQAQWTPQATDLYDLLILTFSVNGKLADLNALKTSSGLSEDDWEALIQYTVQVLSNLVNYKTFGFTKIIPRVDAEKFESVVKASSNADQGSALFTKLKQHIYALSPESALFIGKRKDGHVSNYYLGEPVGDAEVDAIQNVAEKLGVDILNTRVKKNGAGDYTLLVASAKTSPPSVHDFQIDSTPAKLTIEYGDYASSLTKVVAALQEAKQYTANDHQSAMIEGYVKSFNSGSIPEHKAASTEWVKDIGPVVESYIGFVETYVDPYGGRAEWEGFTAIVDKQLSAKYEALVNGAPKLIKSLPWGTDFEVDVFRKPDFTALEVVSFATGGIPAGINIPNYYEVRESTGFKNVSLANILAAKVPNEELTFIHPDDVELYNAWDSRAFELQVANHELLGHGSGKLFQEGADGKLNFDPEKVINPLTGKPITSWYKPGQTPDSVLGEVSSSMEECRAETVALYLVSNLDILKIFNYVDKQDIEDIQYITFLLMARAGLRALEFYDPATKKHGQAHMQARMGITQYLIQAGIARLELIQDANGELENLYVRVDREKVLSKGKEVVGQLLIELQVRKSTADGTGSRDFYTTLTEPISGWEGKIRDIVLKKKLPRKIFVQPNTFVVNGEVQLKEYPLTAAGVIESFIERRL

SEQ ID NO: 2 - protein, 722 aa, Artificial Sequence
  protein encoding section of expression cassette
MKTNLFLFLIFSLLLSLSSAEFMATTTVHRERFLADKSAPLCGMDIRKSFDQLSSKEKLYTHYVTEASWAGARIIQAQWTPQATDLYDLLILTFSVNGKLADLNALKTSSGLSEDDWEALIQYTVQVLSNLVNYKTFGFTKIIPRVDAEKFESVVKASSNADQGSALFTKLKQHIYALSPESALFIGKRKDGHVSNYYLGEPVGDAEVDAIQNVAEKLGVDILNTRVKKNGAGDYTLLVASAKTSPPSVHDFQIDSTPAKLTIEYGDYASSLTKVVAALQEAKQYTANDHQSAMIEGYVKSFNSGSIPEHKAASTEWVKDIGPVVESYIGFVETYVDPYGGRAEWEGFTAIVDKQLSAKYEALVNGAPKLIKSLPWGTDFEVDVFRKPDFTALEVVSFATGGIPAGINIPNYYEVRESTGFKNVSLANILAAKVPNEELTFIHPDDVELYNAWDSRAFELQVANHELLGHGSGKLFQEGADGKLNFDPEKVINPLTGKPITSWYKPGQTPDSVLGEVSSSMEECRAETVALYLVSNLDILKIFNYVDKQDIEDIQYITFLLMARAGLRALEFYDPATKKHGQAHMQARMGITQYLIQAGIARLELIQDANGELENLYVRVDREKVLSKGKEVVGQLLIELQVRKSTADGTGSRDFYTTLTEPISGWEGKIRDIVLKKKLPRKIFVQPNTFVVNGEVQLKEYPLTAAGVIESFIERRLKHDEL

SEQ ID NO: 3 - protein, 5 aa, Artificial Sequence
  ER retention sequence
KHDEL

SEQ ID NO: 4 - protein, 4 aa, Artificial Sequence
  ER retention sequence
KDEL

SEQ ID NO: 5 - DNA, 21 bp, Artificial Sequence
  forward primer specific to the inserted aflatoxin-degrading enzyme sequence
gttggcagatcttaacgctct

SEQ ID NO: 6 - DNA, 21 bp, Artificial Sequence
  reverse primer specific to the inserted aflatoxin-degrading enzyme sequence
cttcccattcagccctacctc